(19) United States
(12) Patent Application Publication
(10) Pub. No.: US 2009/0264971 A1
(43) Pub. Date: Oct. 22, 2009

(54) Dermatological Device for Providing Therapeutic Heat Treatment

(75) Inventor: James C. Wickstead, Mendham, NJ (US)

Correspondence Address: CONNOLLY BOVE LODGE & HUTZ LLP, P.O. BOX 2207, WILMINGTON, DE, 19899, US

(73) Assignee: ESSENTIALS BY AMY LLC, New York, NY (US)

(21) Appl. No.: 12/424,884

(22) Filed: Apr. 16, 2009

Related U.S. Application Data
(60) Provisional application No. 61/124,312, filed on Apr. 16, 2008.

(51) Int. Cl. A61F 7/00

(52) U.S. Cl. 607/108

(57) ABSTRACT

A dermatological device for application of heat treatment to the skin at an optimal temperature range. The dermatological device comprises an outer housing containing a thermal module and an activator. The outer housing comprises upper and lower surfaces, the upper surface comprising a button and the lower surface comprising thermally-conductive material configured for contact with the skin. The thermal module comprises a composition which, upon activation, undergoes an exothermic chemical reaction to generate heat. The activator is disposed within the thermal module and comprises a pliant member coupled to a support. The support displaces the pliant member at a distance from a bottom surface of the thermal module and the pliant member is configured to activate the exothermic chemical reaction when it is depressed or deformed. The pliant member is located at least partially underneath the button of the outer housing such that depressing the button depresses the pliant member.

### CROSS-REFERENCE TO RELATED APPLICATIONS

This application claims priority pursuant to 35 U.S.C. §119(e) to U.S. provisional application Ser. No. 61/124,312, filed Apr. 16, 2008, which is hereby incorporated by reference in its entirety.

### FIELD OF THE INVENTION

The present disclosure relates to cosmetic devices and, more particularly, to dermatological devices for applying heat treatment to the skin.

### BACKGROUND

The elderly population represents the fastest growing population group in the United States. It is therefore no surprise that demand for anti-aging treatment and procedures is increasing at unprecedented rates. As the battle against aging is most commonly fought on the skin, many seek cosmetic surgery or other non-surgical procedures to improve the appearance and texture of their skin for a more youthful look.

The most popular cosmetic surgical procedure is a face lift. Face lifts typically involve the removal of excess facial skin, with or without the tightening of underlying tissues, and the re-draping of the skin on the patient's face and neck. While a face lift may dramatically reduce the signs of aging, it is a serious and invasive procedure that may require general anesthesia. Moreover, the recovery and healing times may take anywhere from a few days to a few weeks.

There are a number of non-surgical procedures to improve the appearance and texture of skin, such as chemical peels, dermabrasion and laser resurfacing. These procedures may be effective for removing fine lines and smoothing out the skin. While these procedures may have the advantages of being non-invasive and requiring relatively short recovery times, multiple treatments are needed to obtain the desired result. Moreover, because these procedures generally involve removing layers of skin, they may thin the skin, which has already thinned significantly from aging.

Both surgical and non-surgical procedures are generally expensive and often painful. A more desirable alternative are in-home skin care treatments that may easily be applied by a user. Many in-home skin care treatments involve the application of medicated serums and emollients to the skin. Oftentimes, such treatments provide limited improvements in the skin's appearance. This may be due, in part, to the way in which the treatment is applied to the skin. As a result, devices have been developed to assist in the application of the various treatments to the skin.

There are various devices that assist in applying various skin formulations and medicaments to the skin. Some devices apply a vibratory massage or electric current to the skin. Other devices may apply heat to the skin. Electrically-powered heating devices often require additional mechanisms for adjusting or regulating temperature so as to prevent overheating and burn to the skin. Many of these devices leave it up to the user to select the temperature to which it is heated. This is undesirable, as the user typically is not knowledgeable as to the optimal or safe temperature ranges that such a device should be set. Moreover, because these devices are generally used in bathrooms, and may potentially be in contact with water from the sink or bathtub, they may malfunction or even pose a risk of electric shock if they come into contact with water.

What is needed, therefore, is a dermatological device which is capable of providing heat to the skin at a reliably regulated temperature that reduces some of the dangers inherent in electrically-powered devices.

### SUMMARY

The dermatological devices disclosed herein provide a safe and effective way for applying moving pressure heat treatment to the skin. The dermatological devices further provide heat treatment at a temperature range that has been found to be optimal for improving the appearance of skin and enhancing absorption of various skin treatments into the skin. Because the dermatological devices utilize an exothermic chemical reaction to generate heat, they do not require electric or battery power for operation and are therefore safe for use in wet environments, such as kitchens and bathrooms. Moreover, the dermatological devices reliably deliver heat at the optimal temperature range for treatment to the skin and to open up skin pores, without the need for a separate mechanism for regulating the temperature as is found in many electrically-powered devices.

A number of chemical compositions may be formulated to provide an exothermic chemical reaction. While the embodiments herein disclose the use of a preferred composition comprising an aqueous solution of sodium acetate, it is understood that any composition may be used so long as it produces an exothermic chemical reaction that generates heat not exceeding 150° F., and preferably 130° F. The exothermic chemical reaction may be activated by a snap or other concussive deformation of a pliable object, such as metal. Once activated, the aqueous sodium acetate solution crystallizes and gives off heat.

The range for heating that provides optimal therapeutic effect on the skin and absorption of formulations is about 100° F. to about 130° F., and more preferably between 110° F. to about 124° F. Heating in these ranges has been found to provide optimal conditions for the uptake of various moisturizers and medicaments to the skin and increasing blood circulation to the skin.

In accordance with one embodiment, a dermatological device comprises a thermal module comprising a casing and a composition disposed within the casing. The composition is configured to undergo an exothermic chemical reaction to generate heat upon activation. An activator disposed within the casing and the activator comprises a pliant member coupled to a support. The pliant member is configured to activate the composition upon deforming. The support is configured to position the pliant member at a distance from said casing and to allow the composition to contact the pliant member.

In accordance with another embodiment, a dermatological device comprises a housing having a resiliently depressable portion. A thermal module is configured to be coupled to the housing and the thermal module comprises a composition configured to undergo an exothermic chemical reaction to generate heat upon activation. An activator is disposed within the thermal module. The activator comprises a pliant member coupled to a support. The pliant member is configured to activate the composition upon deformation.

In a further embodiment, a dermatological device for application of heat treatment to the skin at an optimal temperature range. The dermatological device comprises an outer housing containing a thermal module and an activator. The outer housing comprises upper and lower surfaces, the upper surface comprising a button and the lower surface comprising thermally-conductive material configured for contact with the skin. The thermal module comprises a composition which, upon activation, undergoes an exothermic chemical reaction to generate heat. The activator is disposed within the thermal module and comprises a pliant member coupled to a support. The support displaces the pliant member at a distance from a bottom surface of the thermal module and the pliant member is configured to activate the exothermic chemical reaction when it is depressed or deformed. The pliant member is located at least partially underneath the button of the outer housing such that depressing the button depresses the pliant member.

Other objects, features and advantages of the present invention will become apparent to those skilled in the art from the following detailed description.

### BRIEF DESCRIPTION OF THE DRAWINGS

FIG. 1 is a perspective view of an embodiment of the dermatological device.

FIG. 2A is a side view of an embodiment of the dermatological device.

FIG. 2B is a side view of another embodiment of the dermatological device.

FIG. 3 is a perspective view of an embodiment of the dermatological device.

FIG. 4A is an exploded perspective view of an embodiment of the dermatological device.

FIG. 4B is a top view of an embodiment of the dermatological device. of FIG. 4A with phantom lines to show the activator.

FIG. 4C is a cross-sectional view of the dermatological device of FIG. 4B along the I-I axis.

FIG. 4D is a cross-sectional view of the dermatological device of FIG. 4B along the II-II axis.

FIG. 5A is a perspective phantom view of the heat module, showing the activator.

FIG. 5B is a side phantom view of the heat module of FIG. 5A.

FIG. 6 is a top view of the heat module.

FIG. 7 is an exploded perspective view of an embodiment of the activator.

FIG. 8A is a top view of the support for the activator.

FIG. 8B is a cross-sectional view of the support of FIG. 8A along the I-I axis.

FIG. 8C is a cross-sectional view of the support of FIG. 8A along the II-II axis.

FIG. 9A is a perspective view of an embodiment of the dermatological device.

FIG. 9B is a perspective view of another embodiment of the dermatological device.

FIG. 9C is a perspective view of a further embodiment of the dermatological device.

FIG. 10A is an exploded perspective view of an embodiment of the dermatological device.

FIG. 10B is a top view of an embodiment of the dermatological device. of FIG. 10A

FIG. 10C is a cross-sectional view of the dermatological device of FIG. 10B along the I-I axis.

FIG. 10D is a cross-sectional view of the dermatological device of FIG. 10B along the II-II axis.

FIG. 11A is a top view of an embodiment of the heat module.

FIG. 11B is a side view of the heat module of FIG. 11A.

Like numerals refer to like parts throughout the several views of the drawings.

### DETAILED DESCRIPTION OF THE EMBODIMENTS

A more complete appreciation of the disclosure and many of the attendant advantages may be obtained, as the same becomes better understood by reference to the following detailed description of the exemplary embodiments.

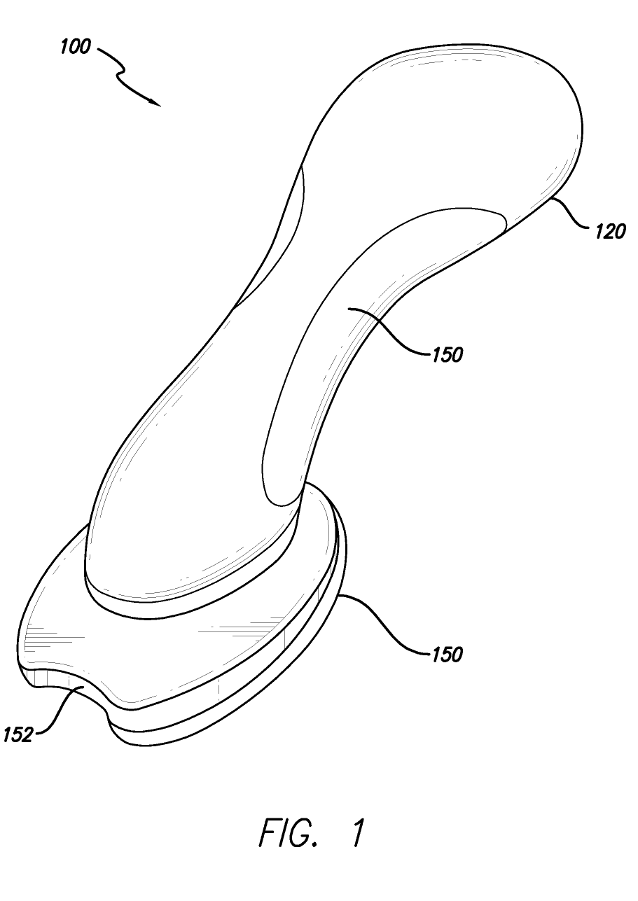

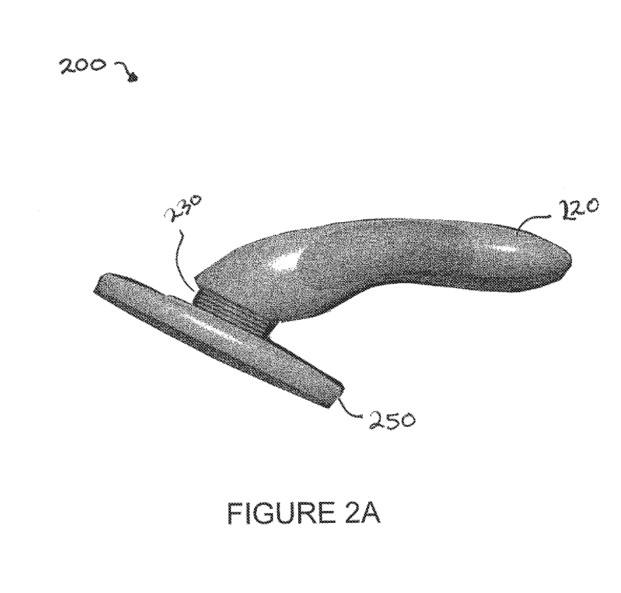

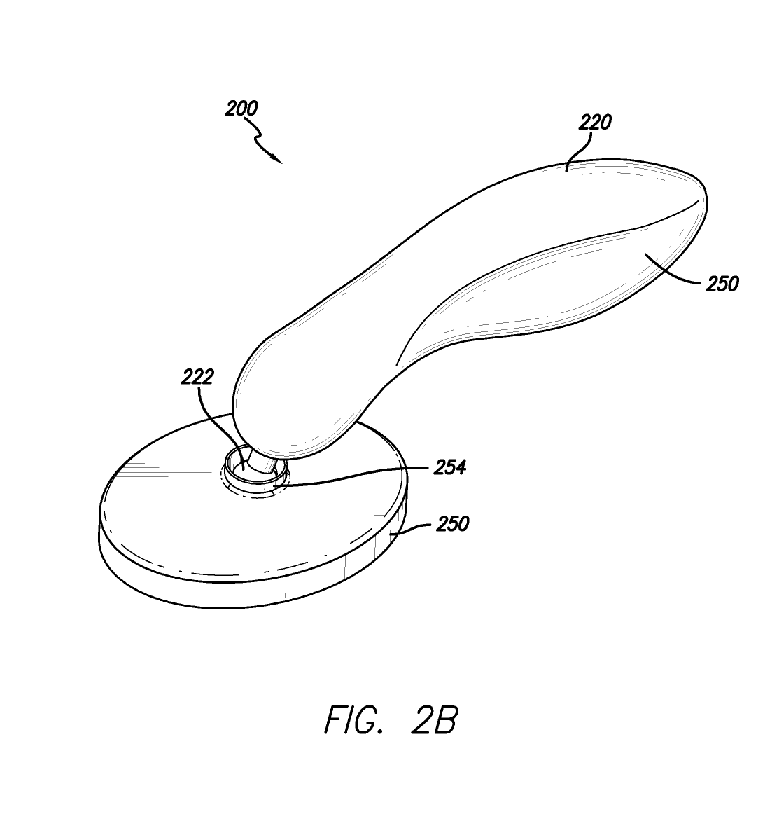

The dermatological devices disclosed herein may be provided in a variety of configurations and sizes, depending on the parts of the body to which treatment is intended. FIG. 1 shows an embodiment of the dermatological device 100 which is suitable for treating the body and the face. The dermatological device 100 includes a handle 120 to assist the user in applying treatment to hard-to-reach areas and a housing 150 that contains the heat module. The handle 120 may be shaped and contoured for a stable handgrip. In a preferred embodiment, the handle 120 is constructed of lightweight plastic and may be provided with a soft grip overlay of foam or rubber 150. The configuration of the handle 120 and the heat module housing 150 may further be configured for application to the face. For example, a concave notch 152 may be provided for the heat module housing 150 for application around the eye. The particulars of the heat module will further be described with respect to the other figures and is equally applicable to the embodiments shown in FIGS. 1 and 2A-B.

FIGS. 2A-2B show embodiments of the dermatological device 200 in which the handle 220 and thermal module 250 are configured to articulate relative to one another by a pivotable attachment. This provides the freedom of movement as the thermal module 250 is applied and moved along the varying contours of the skin surface. In FIG. 2A, the attachment 230 are bellows which permit the thermal module 250 to pivot relative to the handle 220. In FIG. 2B, a ball-and-socket provides the attachment means. The handle 220 is configured with a ball 222 at one end and the thermal module 250 includes a receiving socket 254. The advantage of the ball-and-socket configuration is that the thermal module 250 may be removed and replaced with relative ease. Moreover, the ball-and-socket configuration also provides a mechanism not only for attaching the handle 220 to the heat module 250, but also to activate the heat module to generate heat, as will be further described.

FIG. 3 shows a more compact and hand-held embodiment of the dermatological device 300 comprising a domed hand-held attachment 320 coupled to the thermal module 350. The thermal module 350 is further contoured with a concave portion 352 to permit application of the thermal module 350 around the eyes.

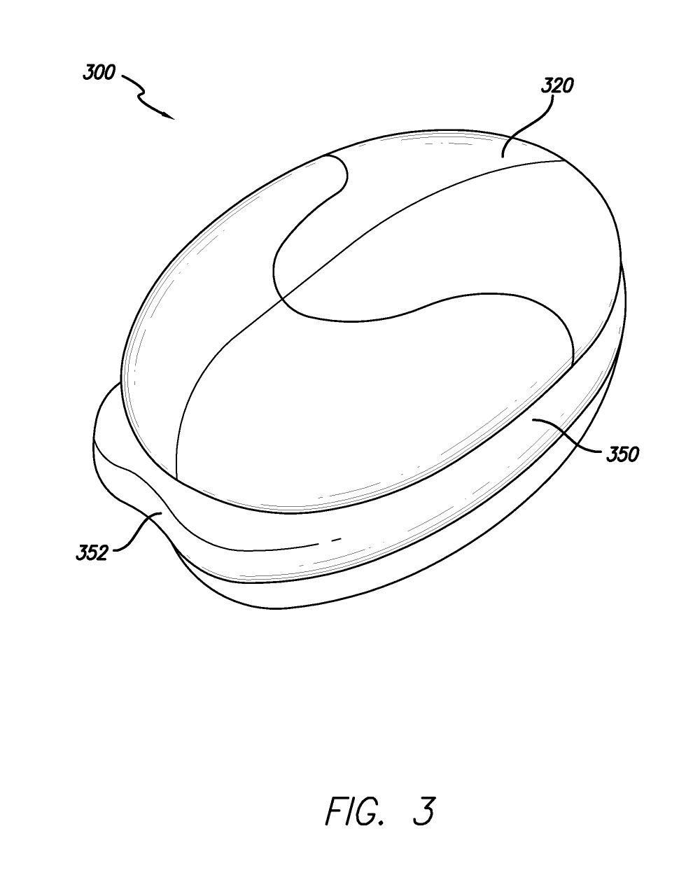

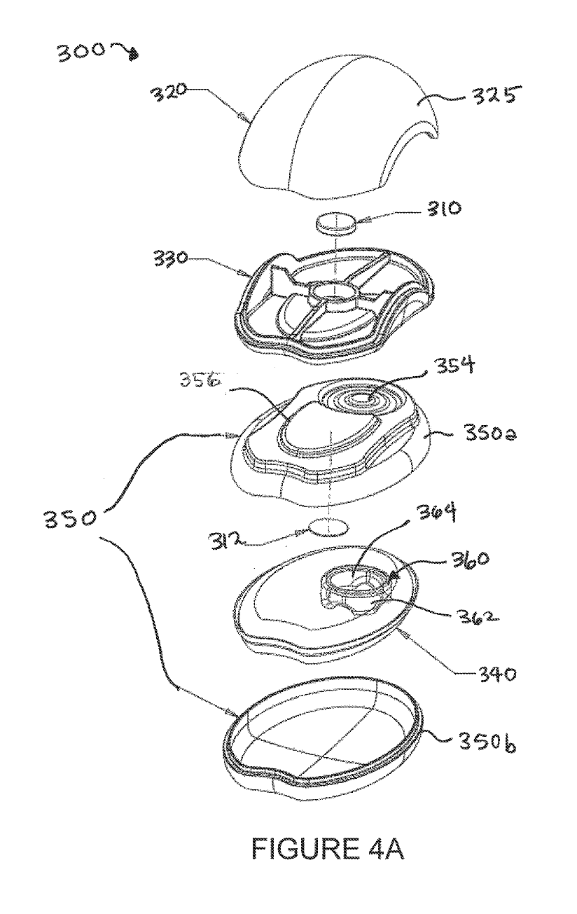

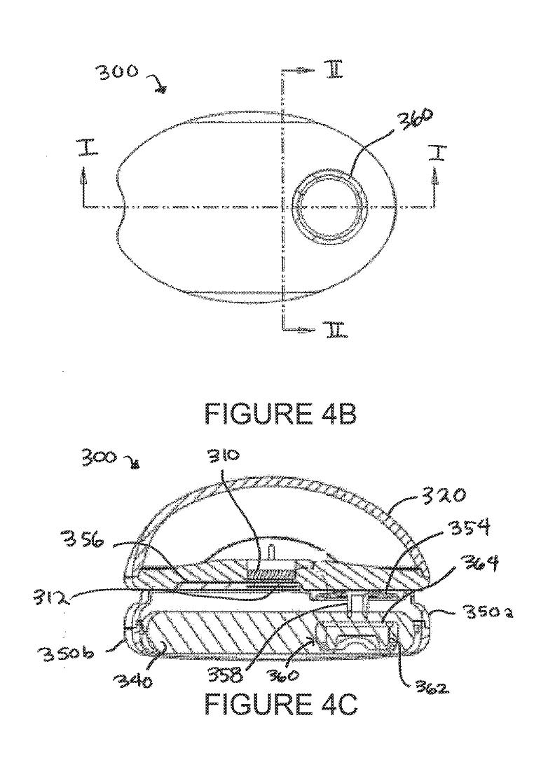

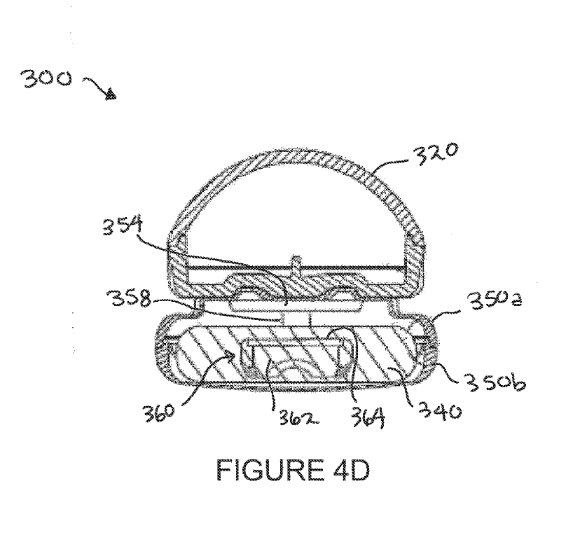

FIGS. 4A-D further illustrate the various components that comprise the dermatological device 300. Referring now to FIG. 4A, the domed hand-held attachment 320 comprises an outer shell 325 and a bottom portion 330. A magnet 310 is disposed within the domed hand-held attachment 320 and is fixed to the bottom portion 330.

A housing 350 is magnetically coupled to the hand-held attachment 320 via a metal or magnetic disk 312 fixed to the housing 350. The housing 350 comprises an upper surface 350a and a lower surface 350b. The housing upper surface 350a is preferably constructed of a rigid material, such as plastic, polypropylene, or the like. The depressable portion 354 of the upper surface 350a, however, is constructed of a resilient, pliable material to permit depression by a finger. The depressable portion 354 may be constructed entirely of a flexible material or provided as a rigid button that is connected to the upper surface 350a via a flexible membrane. The upper surface 350a preferably includes a raised area 356 so as to provide insulation when coupled with the bottom portion 330 of the hand-held attachment 320.

A thermal module 340 is disposed within the housing 350. The thermal module 340 comprises a composition which, upon activation, undergoes an exothermic chemical reaction to generate heat. In a preferred embodiment, the chemical reaction is reversible so as to enable multiple uses of the thermal module 340. In a preferred embodiment, the composition comprises an aqueous solution of sodium acetate, preferably a supersaturated solution of sodium acetate.

An activator 360 is disposed within the thermal module 340 and is in contact with the composition. The activator 360 comprises a support 362 and a pliant member 364 coupled to the support. The activator 360 is configured to activate the exothermic chemical reaction by deformations of the pliant member 364 via applied pressure. The pliant member 364 is preferably made of a metal. The deformation preferably exerts a snap concussion within the aqueous sodium acetate solution which causes the molecules in the liquid to crystallize and solidify. This chemical reaction, in turn, generates controllable heat not exceeding about 135° F. The pliant member 364 may have a dome shape which, upon deformation by pressing on the dome, creates the snap concussion.

The support 362 is preferably made of rigid material and comprises a top end to which the pliant member 364 is attached and scalloped or arched edging along either one or both of the top and bottom ends so as to allow the composition to flow through and contact the pliant member. The support 362 is further configured to displace the pliant member 364 at a distance from the bottom of the thermal module 340 and place it in closer proximity to the depressable portion 354 of the upper surface 350a of the housing 350.

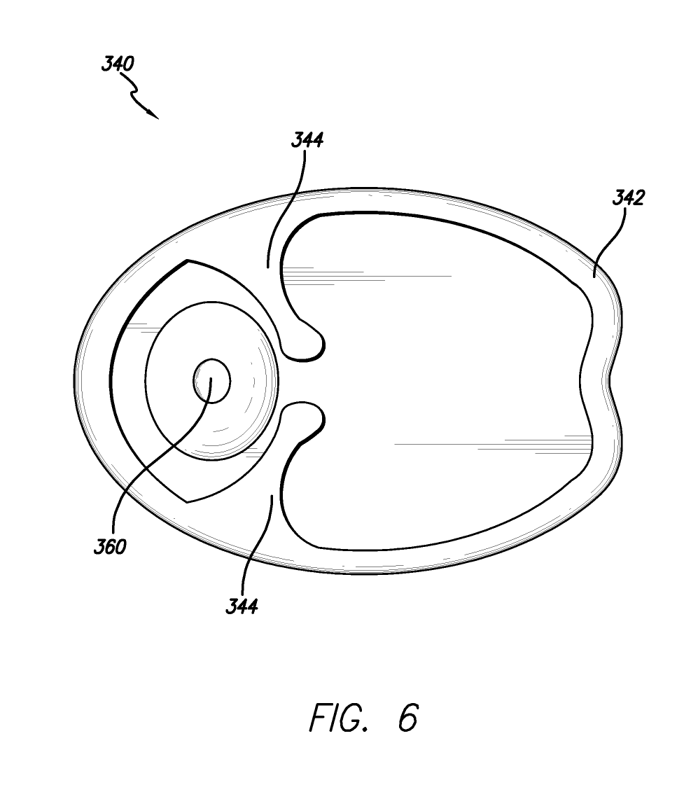

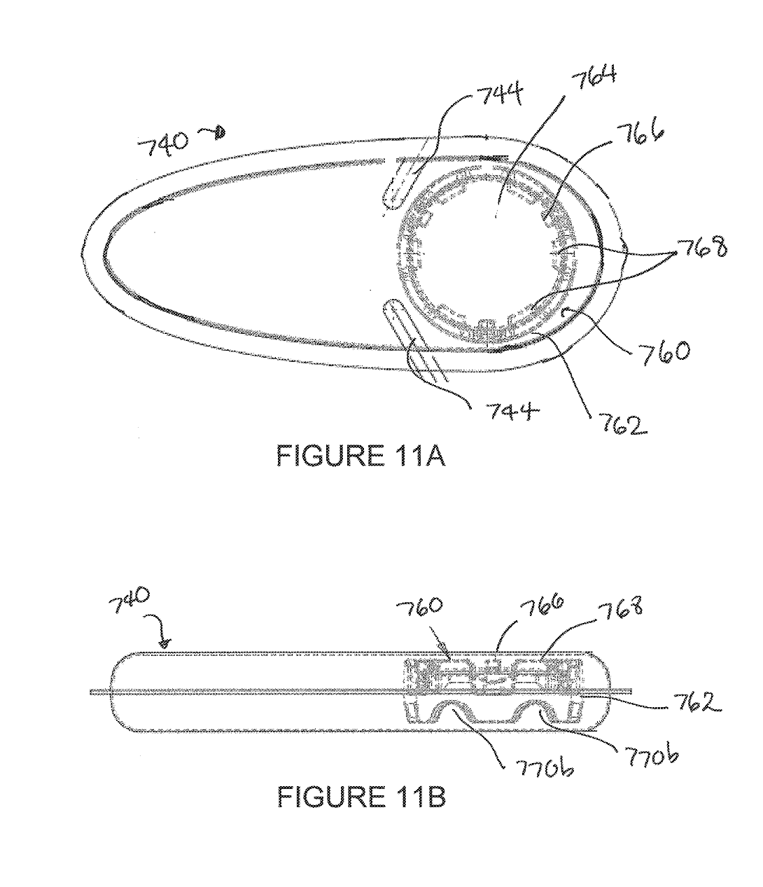

When the dermatological device is completely assembled, the depressable portion 354 is substantially aligned with the activator 360, which is preferably located at a fixed location within the thermal module 340. The location of the activator 360 may be fixed by providing heat seals or other barriers around the activator 360, as shown in FIGS. 6 and 11A. The thermal module 340 may further be configured into a non-symmetric shape so as to ensure placement in the housing 350 in the orientation which would align the depressable portion 354 with the activator 360.

Thus, when the thermal module 340 is contained within the housing 350, the user need only depress or apply pressure with a finger on the depressable portion 354 to engage and deform the pliant member 364 of the activator to initiate the exothermic chemical reaction. This reaction, in turn, will proceed to warm the bottom surface 350b of the housing 350.

In accordance with one embodiment shown in FIG. 2B, the ball-and socket joint may be configured so as to provide both assembly and activation of the exothermic chemical reaction. This may be done, for example, by substantially aligning the receiving socket 254 above the activator 360 such that insertion of the ball portion 222 in the handle 220 will exert pressure onto the pliant member 364 to cause the deforming activation.

The upper and bottom surfaces 350a, b may be further configured to enhance heat transfer from the thermal module 340 onto the bottom surface 350b, which is applied directly to the skin. The upper surface 350a may be provided with metal, chrome plating or other material that reflects heat downward towards the bottom surface 350b. The bottom surface 350b may be made of a thermally conductive material, such as polypropylene or metal, and have a thickness of about 0.25 mm to about 0.75 mm, preferably about 0.4 mm to about 0.6 mm, so as to provide efficient heat transfer. The bottom surface 350b contacting the skin may have a curved smooth surface or a textured surface to retain the formulation or medicament to be applied to the skin. In a preferred embodiment, the texture may comprise grooves or ridges which may retain and warm the formulation or medicament for application to the skin.

FIGS. 4B-D show a fully assembled dermatological device 300. The phantom top view of FIG. 4B depicts the relative location of the activator 360. It is understood that the activator 360 may be located in other areas of the thermal module 340, so long as it may be aligned with the depressable portion 354. As further shown in FIGS. 4C-D, a protruding member 358 is disposed underneath the depressable portion 354 and is in contact with the thermal module 340 directly above the pliant member 364. The protruding member 358 exerts pressure on the pliant member 364 when pressure is applied to the depressable portion 354.

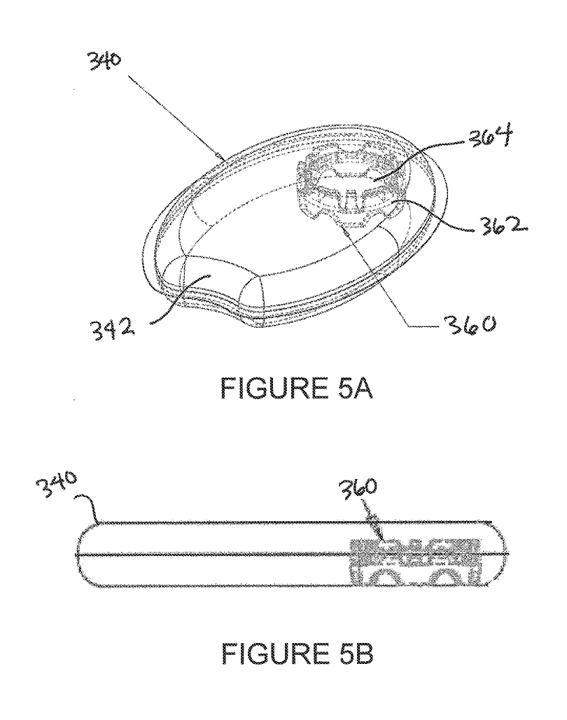

FIGS. 5A-B and 6 illustrate an embodiment of the thermal module 340. The thermal module 340 comprises a casing 342 containing a composition that is capable of undergoing an exothermic chemical reaction to generate heat upon activation. Disposed within the thermal module 340 is the activator 360 comprising a pliant member 364 and a support 362. As shown in FIG. 6, heat seals 344 may be disposed around the activator 360 to fix the location of the activator 360 within the thermal module 340.

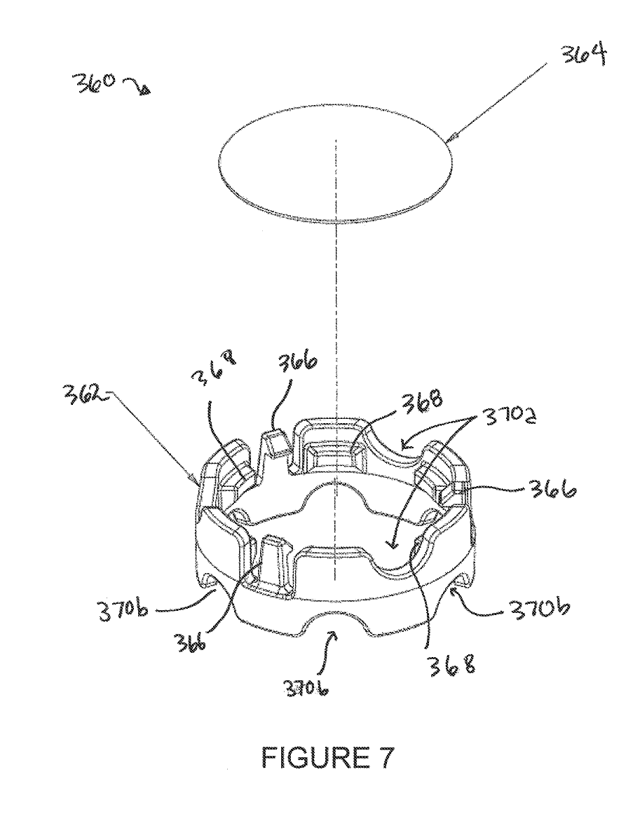

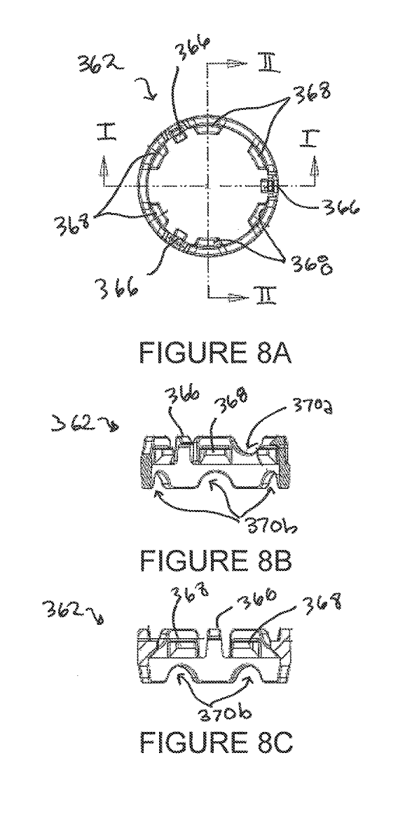

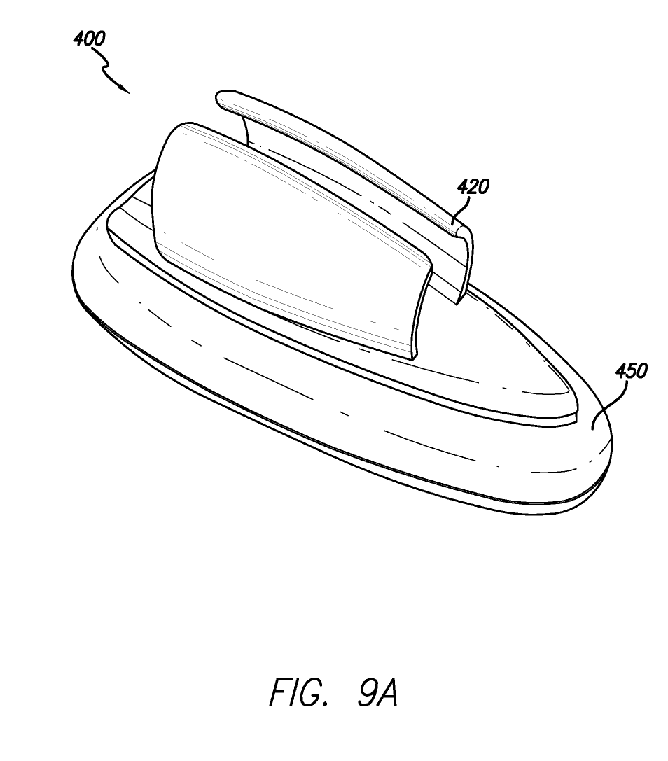

FIGS. 7 and 8A-C further illustrate the structure of the activator 360. The pliant member 364 is preferably a metal or other material that is capable of deformation. To this end, the pliant member 364 may be configured to provide this kind of deformation by application of pressure such as in a dome configuration. The pliant member 364 may also be flat and capable of inverting into the support 362. Any shape of the pliant member 364 is contemplated so long as it is capable of deformation upon the application of ordinary finger pressure. The pliant member 364 is retained by the support 362 through a plurality of hooks 366 disposed on the top end of the support 362. Once the pliant member 364 is hooked in, it rests on a plurality of ledges 368 protruding outwards towards the center of the support 362. The support 362 further comprises a plurality of notches 370a and 370b disposed along the perimeter of the top and bottom ends, respectively. The notches 370a, 370b, permit the flow of the composition through the support so as to contact the pliant member 364. This contact allows the snap deformation of the pliant member 364 to provide an activation of the exothermic chemical reaction.

While pliant member 364 and the support 362 is depicted as having a circular and cylindrical shape, it is understood that the precise shape is not critical and that the structure may be provided in any number of shapes, so long as the pliant member is capable of deforming in a snapping manner sufficient to activate the exothermic chemical reaction by the composition.

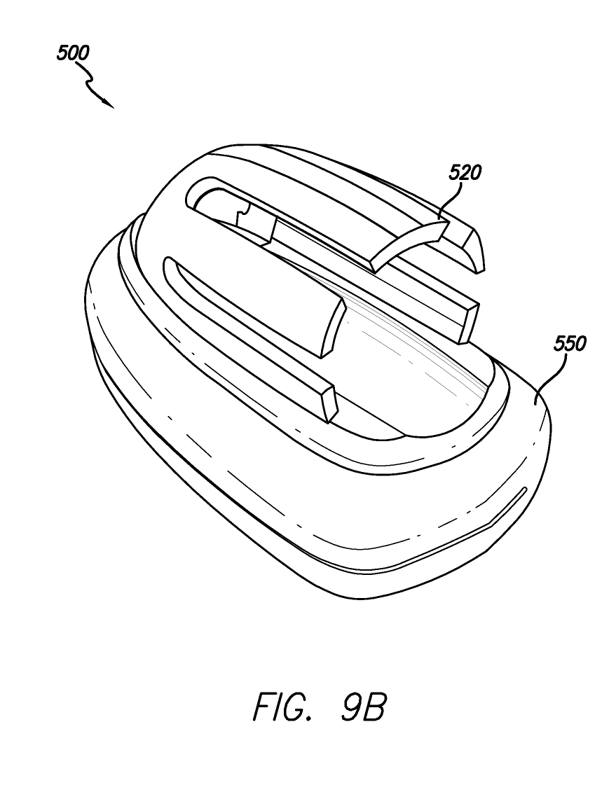

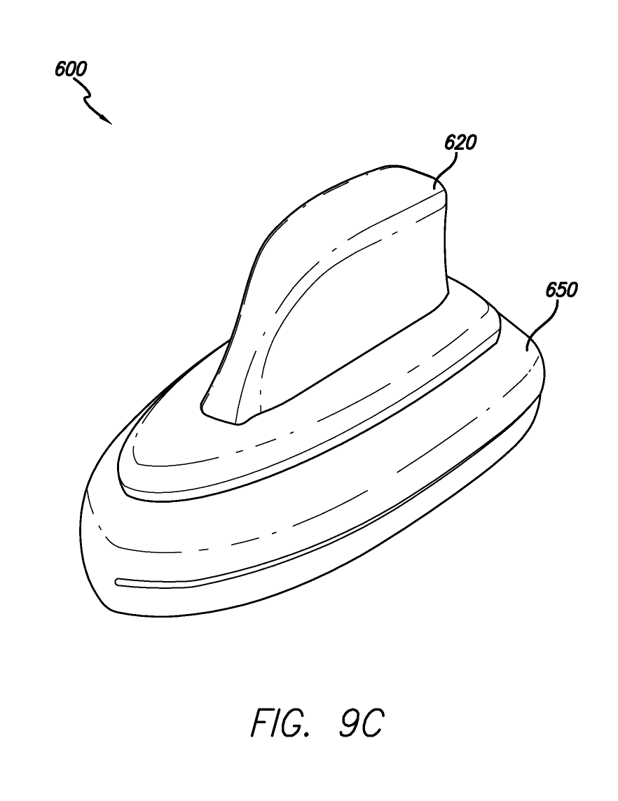

FIGS. 9A-C depict another embodiment of the dematological device 400. FIGS. 9A-B show the dermatological device 400, 500 and 600 as comprising a finger cuff 420, 520 coupled to the thermal module 450, 550. FIG. 9C shows the dermatological device 600 comprising a finger hold 620 coupled to the thermal module 650.

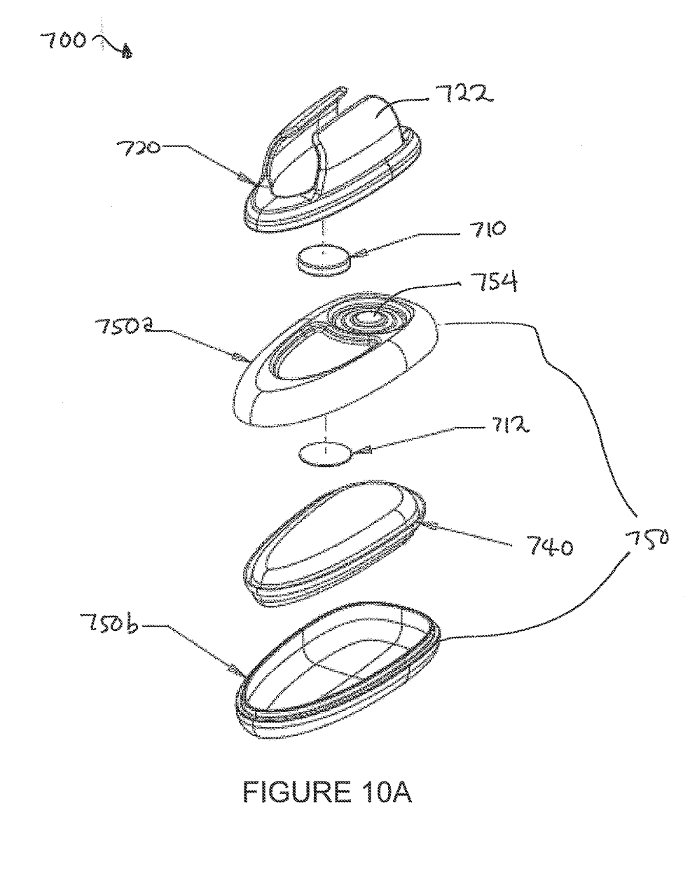

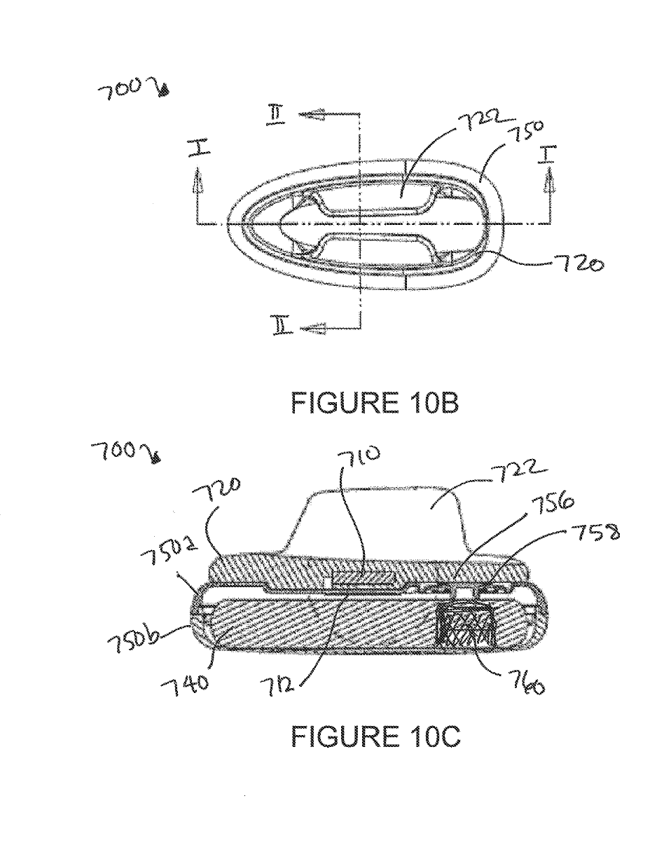

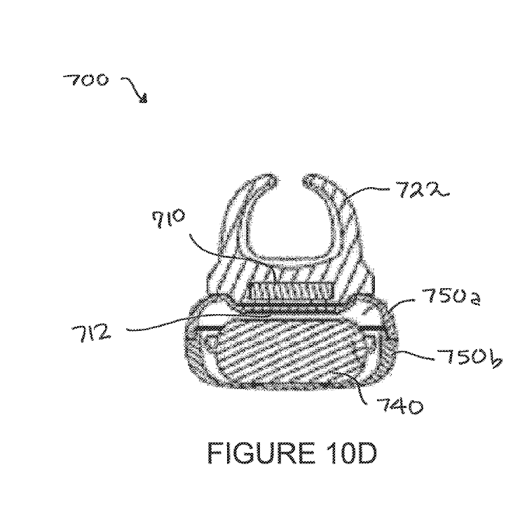

FIGS. 10A-D further illustrate the various components in the dermatological device 700. The dermatological device 700 comprises a finger cuff 722 which is adapted to be worn by a user's finger. The small size of the dermatological device 700 makes it suitable for use on the user's face and on smaller, hard to reach areas. The finger cuff is disposed on base which comprises a magnet 710 to couple to a corresponding magnet or metal plate 712 disposed on the housing upper surface 750a. The housing upper surface 750a further comprises a depressable area 754. A thermal module 740 is disposed within the housing 750. The housing 750 may be configured and constructed in the manner described in connection with FIG. 4A-C.

As further shown in FIGS. 10B-D, a protruding member 758 is coupled to the bottom of the depressable area 754 so as to contact the thermal module 740. The thermal module 740 is more fully depicted in FIGS. 11A-B. The thermal module 740 comprises the composition, the activator 760 and a pair of heat seals 744 to retain the activator 760 in place. Similar to the activator 360 shown in FIGS. 7-8, the activator 760 comprises a support 762 and a snap member 764 retained to the top end of the activator 760 by a plurality of hooks 766 and ledges 768. A plurality of notches 770a and 770b are provided on the top and bottom ends of the support 762 to permit the composition to flow therethrough and contact the snap member 764.

The operation of the dermatological devices disclosed herein may be provided as follows. The user may place the thermal module within the housing and depress the button or depressable area to exert pressure onto the activator. The pliant member of the activator will deform or snap to initiate the exothermic chemical reaction in the thermal module to the desired temperature range of about 100° F. to about 130° F., more preferably of about 100° F. to about 124° F. The housing is then coupled to a hand-held attachment or finger cuff assembly so as to enable handling by the user. The thermal module typically provides substantially continuous heat in the desired range for about 20 to about 30 minutes.

In the preferred embodiment of the thermal module incorporating a sodium acetate aqueous solution, the thermal module may be recharged after each use by boiling or heating the thermal module so as to reverse the crystallization reaction initiated by the activator.

The dermatological device is preferably used in connection with the application of skin medicaments, formulations or serums. In one preferred embodiment, the user may first apply a layer of the skin treatment and subsequently use the heated dermatological device to warm the skin treatment onto the skin. The attendant benefit to this is that both the skin and the skin treatment becomes heated and absorption of the skin treatment into the skin is enhanced. The skin treatments that are most effective in connection with the dermatological devices comprise those which have a relatively low to medium molecular weight and those which are intended for absorption in the skin pores.

In another preferred embodiment, the dermatological device may have a housing lower surface which is specially configured or textured to retain the skin treatment. In accordance with this embodiment, the skin treatment may first be applied to the lower surface and allowed to become heated. Once heated, the dermatological device may be used to apply the treatment to the skin. In one aspect of this embodiment, the housing lower surface may comprise grooves, pits, or a textured surface designed to retain at least a portion of the treatment.

While the embodiments depict the heat module as being provided within a housing, it is understood that the housing is not required for the operation of the dermatological device. The preferred embodiments disclosed herein incorporate a housing for the heat module so as to protect the user for potential leakage of the composition contained in the heat module. However, if the heat module is constructed so as to ensure against harmful leakage, the dermatological devices may be constructed such that the handle, hand-held attachment or finger attachments directly attach to the heat module without an intervening housing. In such embodiments, the heat module may be constructed of a more rigid material so as to support the attachment of the handle, hand-held attachment done or finger attachment via means known in the art, including via bellows or ball-and-socket joints.

Having thus described embodiments of the dermatological device, it should be apparent to those skilled in the art that certain advantages of the dermatological device have been achieved. It should also be appreciated that various modifications, adaptations, and alternative embodiments thereof may be made within the scope and spirit of the present invention.

What is claimed is:

1. A dermatological device comprising:
a thermal module comprising a casing and a composition disposed within the casing, said composition configured to undergo an exothermic chemical reaction to generate heat upon activation; and
an activator disposed within said casing, said activator comprising a pliant member coupled to a support, said pliant member configured to activate said composition upon deforming and said support configured to position said pliant member at a distance from said casing and to allow the composition to contact said pliant member.

2. The dermatological device of claim 1, wherein said pliant member has a dome-shape.

3. The dermatological device of claim 2, wherein said pliant member comprises a metal.

4. The dermatological device of claim 2, wherein said support has a cylindrical shape and said pliant member is coupled to a top end of said support.

5. The dermatological device of claim 4, wherein said top end comprises one or more clips to retain the pliant member.

6. The dermatological device of claim 5, wherein said support has a bottom end having a scalloped edge to allow the composition to pass therethrough.

7. A dermatological device comprising:
a housing comprising a resiliently depressable portion;
a thermal module configured to be coupled to said housing and comprising a composition configured to undergo an exothermic chemical reaction to generate heat upon activation; and
an activator disposed within said thermal module, said activator comprising a pliant member coupled to a support, said pliant member configured to activate said composition upon deformation.

8. The dermatological device of claim 7, wherein said resiliently depressable portion is a push button.

9. The dermatological device of claim 7, wherein said resiliently depressable portion is a flexible and deformable material coupled to said housing.

10. The dermatological device of claim 7, wherein the thermal module further comprises one or more heat seals to position said activator.

11. The dermatological device of claim 10, wherein said activator is configured to be positioned at least partially underneath said resiliently depressable portion when said thermal module is coupled to said housing such that pressure applied to said resiliently depressable portion is transferred to said activator to cause deformation of said pliant member.

12. The dermatological device of claim 7, wherein the exothermic chemical reaction to generate heat is reversible.

13. The dermatological device of claim 12, wherein the composition comprises a supersaturated solution of sodium acetate.

14. A dermatological device comprising:
a housing comprising upper and lower surfaces, said upper surface comprising a depressable area and said lower surface comprising thermally-conductive material;
a thermal module configured to be disposed within said housing, said thermal module comprising a composition which, upon activation, undergoes an exothermic chemical reaction to generate heat; and
an activator disposed within said thermal module, said activator comprising a pliant member coupled to a support, said support configured to displace said pliant member at a distance from a bottom surface of said thermal module.

15. The dermatological device of claim 14 further comprising a hand-held attachment coupled to said housing upper surface.

16. The dermatological device of claim 15, wherein said hand-held attachment and said housing each comprise magnets to couple said hand-held attachment and said housing.

17. The dermatological device of claim 14 further comprising a handle coupled to said housing.

18. The dermatological device of claim 17, wherein said handle and said housing is configured to articulate relative to each other.

19. The dermatological device of claim 17, wherein said handle and said housing are removably coupled together via a ball and socket joint.

20. The dermatological device of claim 19, wherein said handle comprises a ball and said housing comprises a socket located on said housing directly above said depressable area such that coupling said handle having the ball with said housing having the socket depresses the depressable area and activates said composition to undergo the exothermic chemical reaction.

21. The dermatological device of claim 14 further comprising a finger cuff coupled to said housing.
